(19) United States
(12) Patent Application Publication
(10) Pub. No.: US 2009/0088473 A1
(43) Pub. Date: Apr. 2, 2009

(54) METHOD FOR SCREENING OF PROSTAGLANDIN COMPOUNDS COMPRISING AN OPTIMAL FORMULATION FOR THE ENHANCEMENT OF HAIR GROWTH AND THE STIMULATION OF FOLLICULAR ANAGEN AND FORMULATIONS RESULTING THEREFROM

(75) Inventor: Kirk M. Maxey, Ann Arbor, MI (US)

Correspondence Address: REISING, ETHINGTON, BARNES, KISSELLE, P.C., P O BOX 4390, TROY, MI, 48099-4390, US

(73) Assignee: CAYMAN CHEMICAL COMPANY, Ann Arbor, MI (US)

(21) Appl. No.: 12/239,237

(22) Filed: Sep. 26, 2008

Related U.S. Application Data
(60) Provisional application No. 60/975,842, filed on Sep. 28, 2007.

(51) Int. Cl. A61K 31/216; C12Q 1/02; A61K 31/5575

(52) U.S. Cl. 514/530; 435/29; 514/573

(57) ABSTRACT

A method for developing Prostaglandin F_{2α} analogs for enhancing and sustaining the growth of eye lashes. The exemplary method first provides a method for determining the ability of a FP receptor agonist test compound having unknown hair growth ability to induce hair growth in its free acid form. The test compound may then be provided in its amide, or slowly hydrolyzing form and ester form, or rapidly hydrolyzing form to determine the relative hydrolysis rate within corneal tissue. Depending upon its hair growth ability and relative hydrolysis rates, the Prostaglandin F_{2α} analog in one or more forms may be used an ingredient in a binary cosmetic prodrug designed for enhancing and sustaining the growth of eye lashes.

### CROSS REFERENCE TO RELATED APPLICATIONS

The present invention claims priority from U.S. Provisional Application No. 60/975,842, filed on Sep. 27, 2007, and entitled “A Method for Screening of Prostaglandin Compounds Comprising an Optimal Formulation for the Enhancement of Hair Growth and the Stimulation of Follicular Anagen.”

### TECHNICAL FIELD OF THE INVENTION

The field to which the disclosure relates includes methods for identifying compounds for their ability to stimulate hair growth, compositions comprising one or more of said compounds, and methods for stimulating hair growth and changing hair pigmentation in mammals with said compositions. More specifically, the field to which the disclosure relates includes methods for identifying compounds for their ability to stimulate hair growth, compositions comprising one or more said compounds, and methods of topical administration for stimulating eyelash growth and changing eyelash pigmentation.

### BACKGROUND OF THE INVENTION

Opthalmological compositions of analogs of prostaglandin F_{2α} (PGF_{2α}), for example, Latanoprost, Bimatoprost, and Travoprost, may be used as topical treatments for intraocular pressure associated with glaucoma.

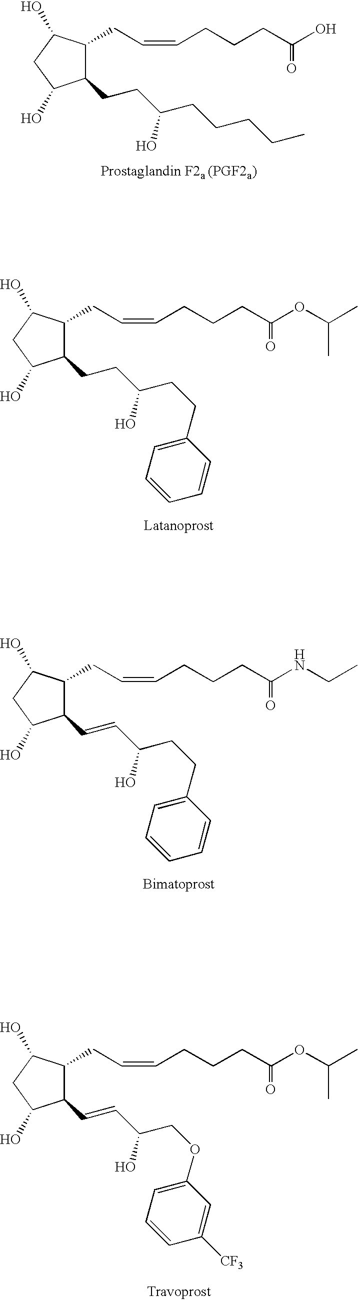

The precise mechanism by which prostaglandins lower intraocular pressure is not certain, but it is believed to be mediated by Prostaglandin FP receptors. Ester and amide derivatives of the active carboxylic acid form of PGF_{2α} analogs have been studied for their pharmacokinetic properties in permeation and bioconversion models (Chien, D. S., Tang Liu, D. D., Woodward, D. F., Journal of Pharmaceutical Science, 86 (10), 1997, 1180-1186).

Hypertrichosis, a darkening, lengthening, and thickening of the eyelashes, is a common side effect in patients receiving topical ocular treatment of glaucoma with analogs of Prostaglandin F_{2α}, including latanoprost, bimatoprost, and travoprost (Husband, A., Worsley, A., Hospital Pharmacist, 12, 2005, 255-261; Feletti, F., Vincenzi, C., et al., Journal of the European Academy of Dermatology, 21 (3), 2007, 421-423; Tosti, A., Pazzaglia, M., et al., Journal of the American Academy of Dermatology, 51 (5), 2004, S149-S150). The precise mechanism by which this side effect occurs is not well understood; however, localized hypertrichosis may be desirable for persons wanting to enhance the length and appearance of their natural eyelashes, eyebrows, scalp hair, and other dermal appendages.

Amide and ester derivatives of potent PGF_{2α} carboxylic acid analogs may be designed for topical pharmacokinetic properties that optimize localized stimulation of hair growth. Amide and ester derivatives of the potent PGF_{2α} carboxylic acid analogs may act as prodrugs. A Review, Prodrugs: Design and Clinical Applications, by Rautio, J., Kumpulainen, H., et al., Nature Reviews Drug Discovery, 7, 2008, 255-270 gives an overview of the design and utility of prodrugs.

### SUMMARY OF THE INVENTION

One exemplary embodiment may be a method that measures the ability of a test compound to increase inositol triphosphate release in cells transfected with the human F_{2α} (FP) receptor.

Another exemplary embodiment of the invention may be a method for determining the rate of conversion of a prodrug derivative of an active PGF_{2α} analog to the parent carboxylic acid compound.

Also in accordance with the exemplary method described above, the test compound may be a FP receptor agonist.

Also in accordance with the exemplary method described above, the test compound may be an FP receptor agonist selected from Latanoprost, Bimatoprost, and Travoprost.

Also in accordance with the exemplary method described above, the test compound may be an ester or amide derivative of one of the group of carboxylic acid-containing compounds (Table 1, wherein X is OH) consisting of:

TABLE 1

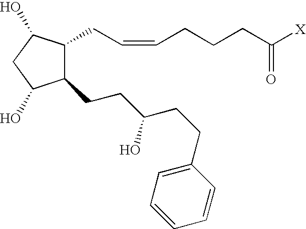

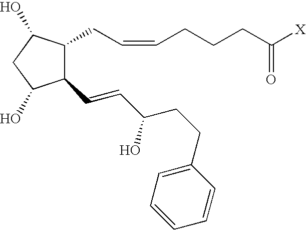

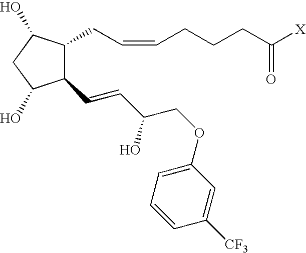

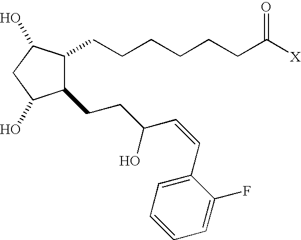

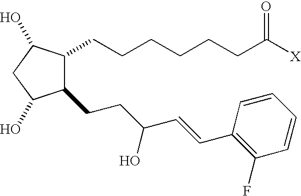

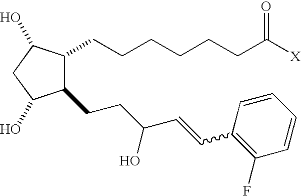

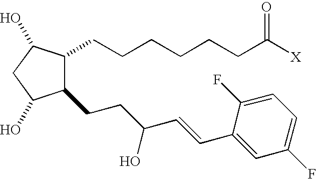

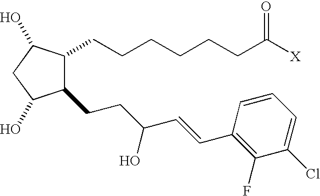

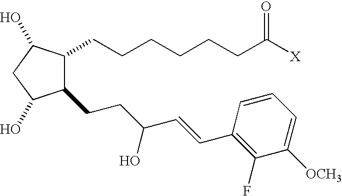

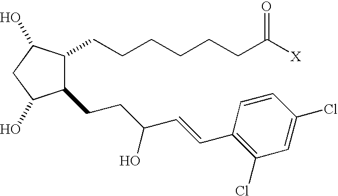

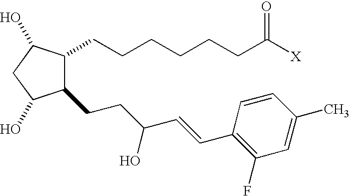

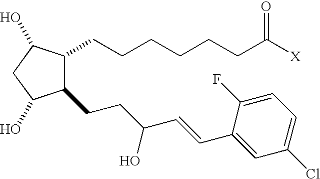

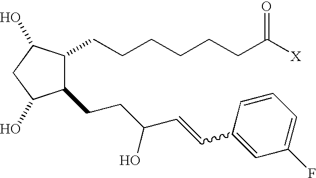

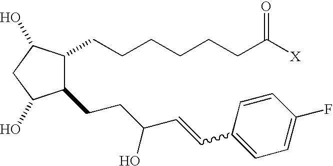

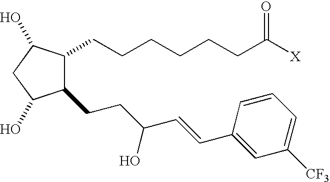

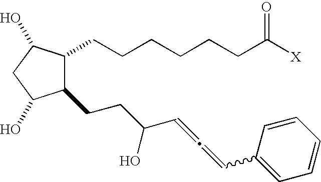

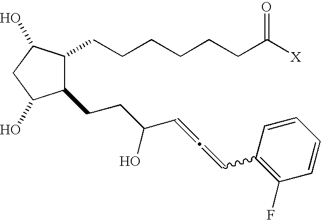

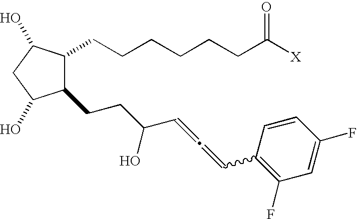

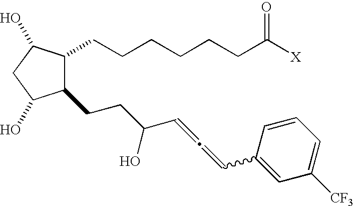

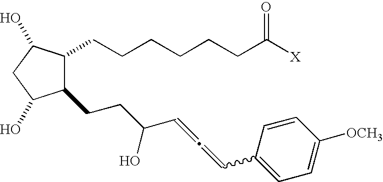

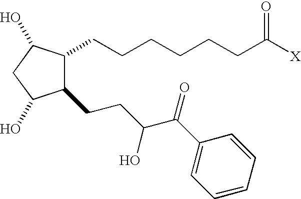

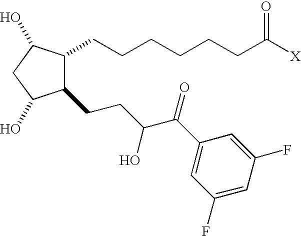

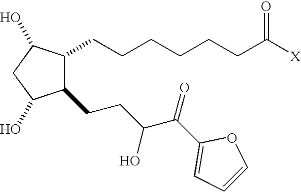

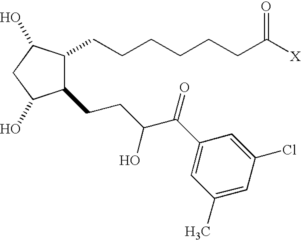

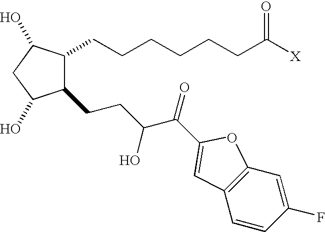

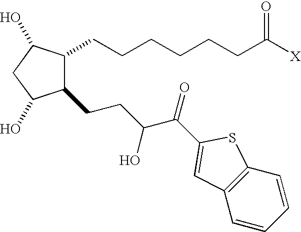

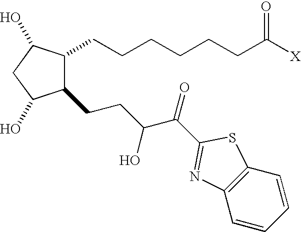

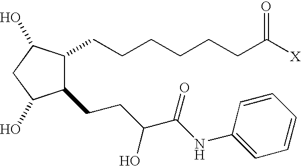

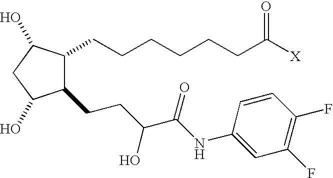

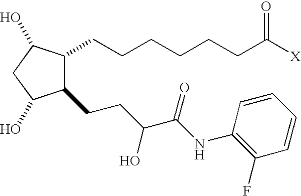

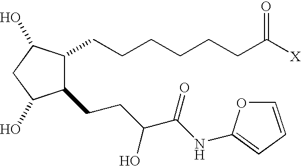

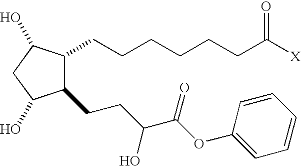

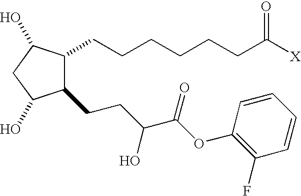

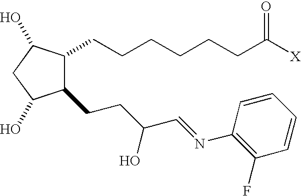

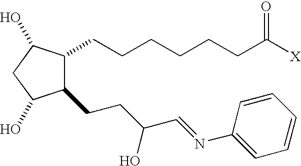

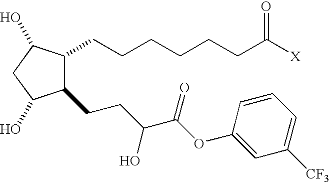

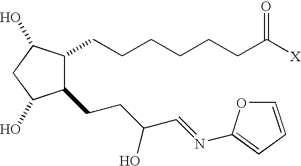

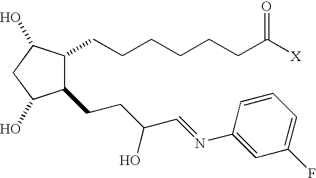

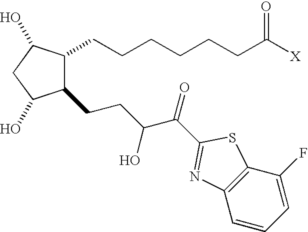

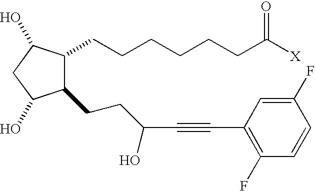

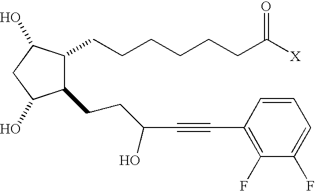

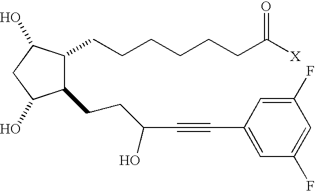

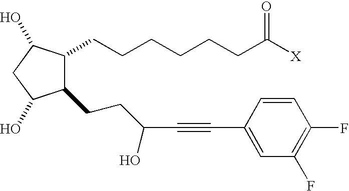

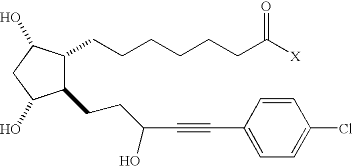

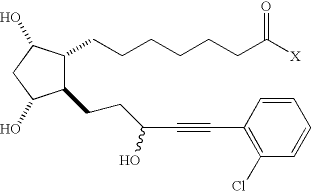

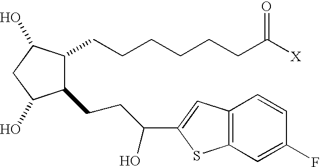

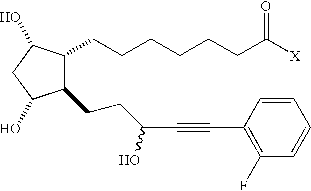

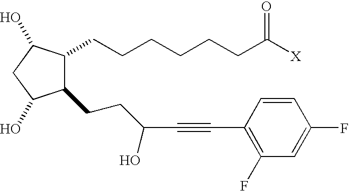

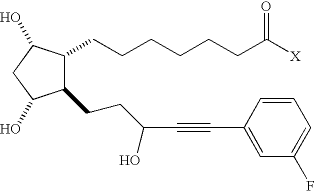

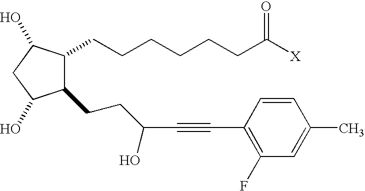

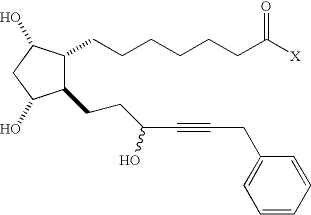

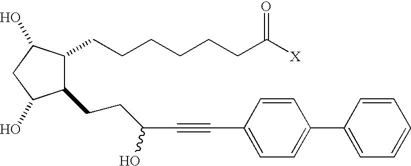

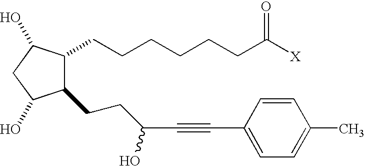

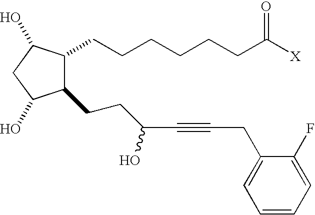

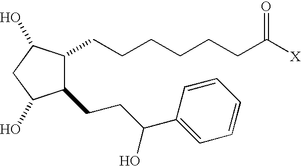

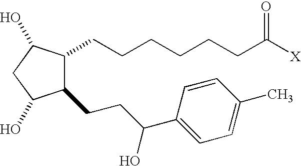

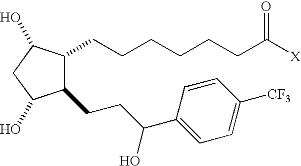

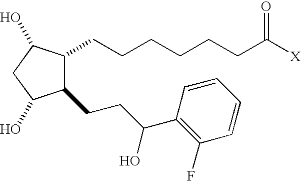

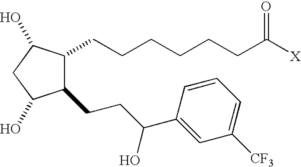

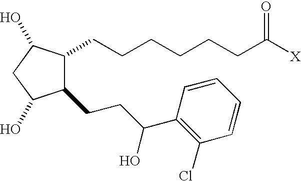

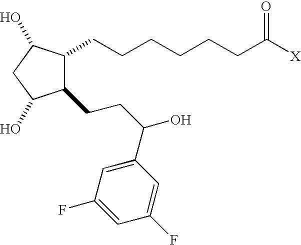

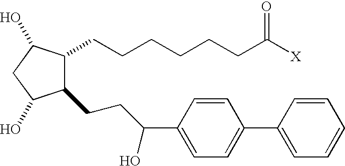

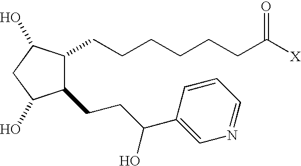

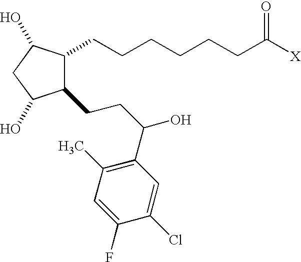

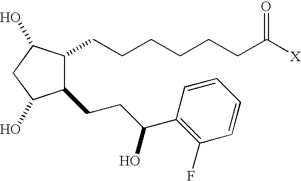

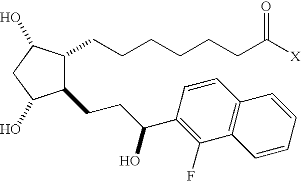

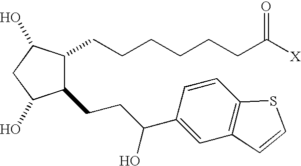

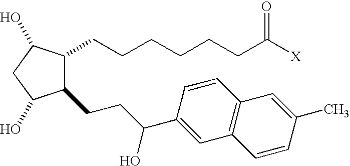

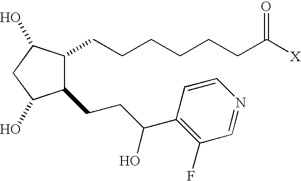

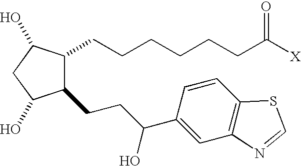

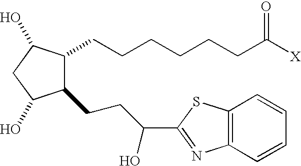

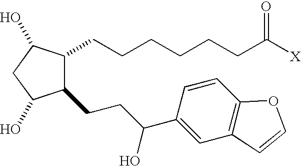

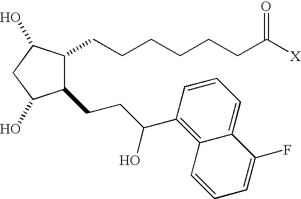

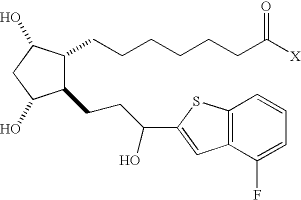

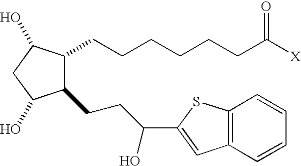

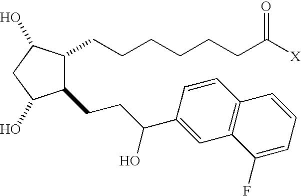

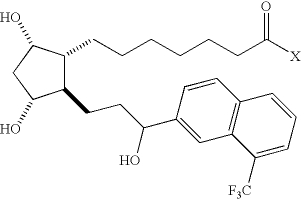

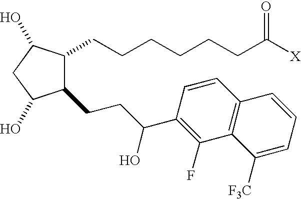

Another exemplary embodiment of the invention may be a method that measures the ability of a mixture comprising at least two test compounds to increase inositol triphosphate release in cells transfected with the human F_{2α} (FP) receptor.

Also in accordance with the exemplary method described above, the mixture of test compounds may comprise two FP receptor agonists.

Also in accordance with the exemplary method described above, the mixture of test compounds may comprise any two FP receptor agonists of the group consisting of Latanoprost, Bimatoprost, and Travoprost.

Also in accordance with the exemplary method described above, the mixture of test compounds may comprise any ester or amide derivative of one or more of the group of carboxylic acid-containing compounds disclosed in Table 1 (wherein X is OH).

Another exemplary embodiment may be a composition comprising any one or more of said test compounds described above.

Another exemplary embodiment may be a method for administering one of the said compositions described in the paragraph immediately above for the treatment of hair loss.

Another exemplary embodiment may be a method for administering one of the said compositions described two paragraphs above for the stimulation of eyelash growth.

Another exemplary embodiment may be a method for administering one of the said compositions described three paragraphs above for the changing of hair pigmentation.

Also in accordance with the above advantages, an exemplary method may be described for identifying combinations of slowly hydrolyzed prodrugs and rapidly hydrolyzed prodrugs of FP receptor agonists. The exemplary method includes an assay employed that may make use of prostaglandin esterase activity within bovine corneal tissue to convert relatively inactive prodrug amide or ester forms of FP receptor agonists to the active free acid forms. Combinations of the slowly hydrolyzed prodrugs and rapidly hydrolyzed prodrugs may be correlated with FP receptor activation as determined using the cell-based FP receptor activation model. Structure activity correlation may be made for each compound in relation to the ability of bimatoprost, formulated as an amide or as an ester, which may enhance eye lash growth.

### DESCRIPTION OF THE DRAWINGS

FIG. 1 shows the chemical structure of bimatoprost as the free acid, the methyl amide, and as the isopropyl ester; and

FIG. 2 displays results of dose-dependent FP receptor stimulation in HEK 293 cells by various prostaglandin analogs in accordance with one exemplary embodiment of the invention.

### DETAILED DESCRIPTION OF THE INVENTION

An exemplary assay method may be described that correlates the ability of test compounds (FP receptor agonists) to induce hair growth with the ability of an established FP receptor agonist, in this case Bimatoprost free acid, to activate the FP receptor. The dose-dependent release of Inositol triphosphate by test compounds compared to the dose-dependent response of Bimatoprost free acid, an established eye lash-enhancing compound, may provide a structure activity relationship that may be used to predict the degree to which a given compound will promote eye lash growth.

The exemplary assay employed for this analysis may make use of a previously described method (P. Kunapuli et al./The Journal of Biological Chemistry, Vol. 272, No. 43, Issue of October 24, pp. 27147-27154 (1997) to measure total intracellular inositol phosphate (InsP) accumulation following stimulation of HEK 293 cells which have been stably-transfected with the human FP receptor. The FP receptor may bind to and may be activated by the free acid forms of PGF_{2α} and PGF_{2α} analogs. Compounds containing ester or amide substituents at the C-1 COOH group are generally inactive at the FP receptor. Therefore, all assays performed according to an exemplary embodiment may utilize the free acid forms of the test compound.

FIG. 2 displays results of dose-dependent FP receptor stimulation in HEK 293 cells by various prostaglandin analogs utilizing the exemplary test method described in the previous paragraph. According to FIG. 2, the 16-phenoxy tetranor PGF_{2α} analog showed the highest dose-dependent release of inositol triphosphate, similar to the free acid form of Bitamoprost, and thus correlates to the highest ability to induce the growth of human hair. Further, (+) fluprestonol illustrated the lowest dose-dependent release of inositol triphosphate, and thus would be predicted to have the lowest ability among the prostaglandin analogs tested to induce hair growth.

The exemplary embodiments also describe binary mixtures of slowly hydrolyzed prodrugs and rapidly hydrolyzed prodrugs of FP receptor agonists. Literature precedence (M. Abramowitz et al./Biochimica et Biophysica Acta 1483 (2000) 285, 293; B. Rasul et al./Survey of Opthamology Vol. 41, Supp. 2, February 1997 S47, S 52; and N. Sharif et al./Journal of Ocular Pharmacology and Therapeutics, Vol. 19, No. 6, 2003) may indicate that the FP receptor binds to and may be activated by the free acid forms of PGF_{2α} and PGF_{2α} analogs as typified by Latanoprost, Travoprost, and Bimatoprost and as shown in Table 1 in the Summary of the Invention above. A prodrug, by definition, is a drug that is administered in a less active or inactive form and converted to the active form in vivo. Substantially reduced FP receptor activity may be observed with ester or amide forms of the compounds.

Slowly hydrolyzing prodrugs typically may have the form of amides containing methyl, ethyl, isopropyl, or other similar substituents of the parent PGF_{2α} analogs. Rapidly hydrolyzing prodrugs typically may have the form of esters containing methyl, ethyl, isopropyl, glyceryl, or similar substituents of the parent PGF_{2α} analogs.

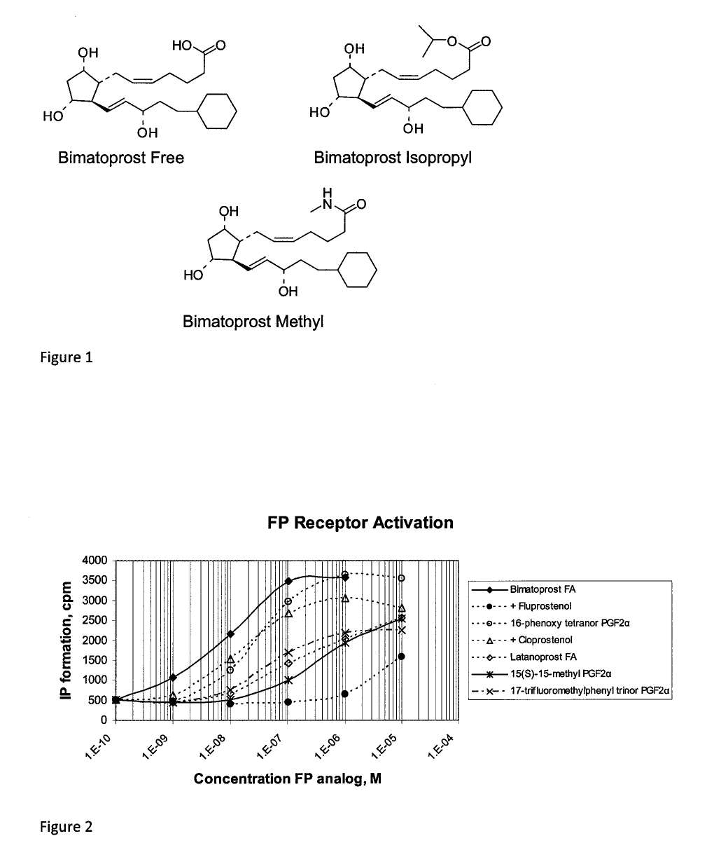

The exemplary binary mixture of slowly and rapidly hydrolyzing prodrugs of PGF_{2α} analogs thus may provide a means to provide sustained activity for optimal hair growth. Representative prodrug derivatives of such compounds may be shown in FIG. 1, which illustrates the chemical structure of Bimatoprost as the free acid, the methyl amide of Bimatoprost, and Bimatoprost as the isopropyl ester. As such, the methyl amide may represent a slowly hydrolyzed prodrug of Bimatoprost and the isopropyl ester may represent a rapidly hydrolyzed prodrug of Bimatoprost.

In addition, esterases within the cornea may hydrolyze the esters and amides to release the active free acid form of the FP receptor agonist compound. Activity of the esterase may be much faster towards esters compared to amide modifications of the free acid agonists. The assay employed by the exemplary embodiments may make use of prostaglandin esterase activity within bovine corneal tissue to convert relatively inactive amide or ester forms of FP receptor agonists to the active free acid forms.

In this assay, amide and ester prodrug derivatives of FP receptor agonists may be incubated with bovine cornea in a buffered, aqueous environment for pre-defined periods of time. At fixed time points, the aqueous solutions may be extracted and then analyzed by HPLC or LC/MS for the presence of the parent ester or amide compound as well as the free acid product of the hydrolysis reaction. Combinations of the slowly and rapidly hydrolyzed prodrug compounds may be correlated with FP receptor activation as determined using the cell-based FP receptor activation model. Structure activity correlation may be made for each compound in relation to the ability of Bimatoprost, derivatized as an amide or as an ester, to enhance eye lash growth.

Thus, an exemplary method for developing Prostaglandin F_{2α} analogs for enhancing the growth of eye lashes may be provided. The exemplary method may first determine the ability of a FP receptor agonist test compound having unknown hair growth ability. Depending upon the hair growth ability of the test compound in its free acid, amide or ester form, the test compound may then be utilized as an ingredient in a binary cosmetic formulation designed for enhancing and sustaining the growth of eye lashes.

While particular exemplary embodiments have been shown and described, numerous variations or alternate embodiments will occur to those skilled in the art. Accordingly, it is intended that the exemplary methods, compositions, and formulations be limited only in terms of the appended claims.

What is claimed is:

1. A method for determining the ability of a FP receptor agonist test compound to induce hair growth, the method comprising:
providing the FP receptor agonist test compound in a free acid form;
providing a culture of human FP-receptor transfected embryonic kidney 293 cells;
introducing the FP receptor agonist test compound to said culture;
measuring total intracellular inositol phosphate accumulation following stimulation of said culture of human FP-receptor transfected embryonic kidney 293 cells; and
comparing said measured total intracellular phosphate accumulation with a measured accumulation of total intracellular phosphate accumulation from a known FP receptor agonist.

2. A method for determining a cosmetic prodrug of Prostaglandin F_{2α} analogs for enhancing and sustaining the growth of eye lashes, the method comprising:
(a) determining the ability of a FP receptor agonist test compound to induce hair growth by:
providing the FP receptor agonist test compound in a free acid form;
providing a culture of human FP-receptor transfected embryonic kidney 293 cells;
introducing said FP receptor agonist test compound to said culture;
measuring total intracellular inositol phosphate accumulation following stimulation of said culture of human FP-receptor transfected embryonic kidney 293 cells; and
comparing said measured total intracellular phosphate accumulation with a measured accumulation of total intracellular phosphate accumulation from a known FP receptor agonist;
(b) repeating step (a) for each additional FP receptor agonist test compound;
(c) determining the relative ability of each of said FP receptor agonist test compounds to induce hair growth by comparing each of said measured total intracellular phosphate accumulations;
(d) selecting an FP receptor agonist test compound having a desired hair growth in said free acid form;
(e) providing said selected FP receptor agonist test compound in an amide form and in an ester form;
(f) determining the hydrolysis rate of said selected FP receptor agonist test compound from said amide form to said free acid form;
(g) determining the hydrolysis rate of said selected FP receptor agonist test compound from said ester form to said free acid form;
(h) repeating steps (e) through (g) for each additional selected FP receptor agonist test compound having a desired hair growth in said free acid form; and
(i) selecting at least one of said selected FP receptor agonist test compounds in said amide form having a first desired hydrolysis rate with at least one of said selected FP receptor agonist test compounds in said ester form having a second desired hydrolysis rate to form a binary prodrug, said first desired hydrolysis rate being slower than said second desired hydrolysis rate.

3. A cosmetic prodrug formed in accordance with the method of claim 2 and comprising at least one of said FP receptor agonist test compounds in said amide form and at least one of said FP receptor agonist test compounds in said ester form.

4. The cosmetic prodrug of claim 3, wherein said FP receptor agonist test compounds in said amide form is selected from the group consisting of an amide containing methyl substituent of a parent PGF_{2α} analog, an amide containing ethyl substituent of a parent PGF_{2α} analog, an amide containing isopropyl substituent of a parent PGF_{2α} analog, and mixtures thereof.

5. The cosmetic prodrug of claim 3, wherein said FP receptor agonist test compounds in said ester form is selected from the group consisting of an ester containing methyl substituent of a parent PGF_{2α} analog, an ester containing ethyl substituent of a parent PGF_{2α} analog, an ester containing isopropyl substituent of a parent PGF_{2α} analog, an ester containing glyceryl substituent of a parent PGF_{2α} analog, and mixtures thereof.

6. The cosmetic prodrug of claim 4, wherein said FP receptor agonist test compounds in said ester form is selected from the group consisting of an ester containing methyl substituent of a parent PGF_{2α} analog, an ester containing ethyl substituent of a parent PGF_{2α} analog, an ester containing isopropyl substituent of a parent PGF_{2α} analog, an ester containing glyceryl substituent of a parent PGF_{2α} analog, and mixtures thereof.

7. The cosmetic prodrug of claim 3, wherein said FP receptor agonist test compounds in said amide form is selected from the group consisting of Bimatoprost in said amide form, Latanoprost in said amide form, Travoprost in said amide form, and mixtures thereof.

8. The cosmetic prodrug of claim 3, wherein said FP receptor agonist test compounds in said ester form is selected from the group consisting of Bimatoprost in said ester form, Latanoprost in said ester form, Travoprost in said ester form, and mixtures thereof.

9. The cosmetic prodrug of claim 7, wherein said FP receptor agonist test compounds in said ester form is selected from the group consisting of Bimatoprost in said ester form, Latanoprost in said ester form, Travoprost in said ester form, and mixtures thereof.

10. A method for determining a cosmetic prodrug of Prostaglandin F_{2α} analogs for enhancing and sustaining the growth of eye lashes, the method comprising:
(a) determining the ability of a FP receptor agonist test compound to induce hair growth by:
providing the FP receptor agonist test compound in a free acid form;
providing a culture of human FP-receptor transfected embryonic kidney 293 cells;
introducing said FP receptor agonist test compound to said culture;
measuring total intracellular inositol phosphate accumulation following stimulation of said culture of human FP-receptor transfected embryonic kidney 293 cells; and
comparing said measured total intracellular phosphate accumulation with a measured accumulation of total intracellular phosphate accumulation from a known FP receptor agonist;
(b) repeating step (a) for each additional FP receptor agonist test compound;
(c) determining the relative ability of each of said FP receptor agonist test compounds to induce hair growth by comparing each of said measure total intracellular phosphate accumulations;
(d) selecting an FP receptor agonist test compound having a desired hair growth in said free acid form;
(e) providing said selected FP receptor agonist test compound in a slowly hydrolyzing form and in a rapidly hydrolyzing form;
(f) determining the hydrolysis rate of said selected FP receptor agonist test compound from said slowly hydrolyzing form to said free acid form;
(g) determining the hydrolysis rate of said selected FP receptor agonist test compound from said rapidly hydrolyzing form to said free acid form;
(h) repeating steps (e) through (g) for each additional selected FP receptor agonist test compound having a desired hair growth in said free acid form; and
(i) selecting at least one of said selected FP receptor agonist test compounds in said slowly hydrolyzing form having a first desired hydrolysis rate with at least one of said selected FP receptor agonist test compounds in said rapidly hydrolyzing form having a second desired hydrolysis rate to form a binary prodrug, said first desired hydrolysis rate being slower than said second desired hydrolysis rate.

11. A cosmetic prodrug formed in accordance with the method of claim 10 and comprising at least one of said FP receptor agonist test compounds in said slowly hydrolyzing form and at least one of said FP receptor agonist test compounds in said rapidly hydrolyzing form.

12. The method of claim 10, wherein said slowly hydrolyzing form of said FP receptor agonist test compound comprises amides of a parent PGF_{2α} analog.

13. The method of claim 12, wherein said amides of a parent PGF_{2α} analog are selected from the group consisting of methyl amides of a parent PGF_{2α} analog, ethyl amides of a parent PGF_{2α} analog, isopropyl amides of a parent PGF_{2α} analog, and other similar substituent amides of a parent PGF_{2α} analog.

14. The method of claim 10, wherein said rapidly hydrolyzing form of said FP receptor test compound comprises esters of said parent PGF_{2α} analog.

15. The method of claim 12, wherein said rapidly hydrolyzing form of said FP receptor test compound comprises esters of said parent PGF_{2α} analog

16. The method of claim 14, wherein said esters of said parent PGF_{2α} analog are selected from the group consisting of methyl esters of said parent PGF_{2α} analog, ethyl esters of said parent PGF_{2α} analog, isopropyl esters of said parent PGF_{2α} analog, glyceryl esters of said parent PGF_{2α} analog, and other similar substituent esters of said parent PGF_{2α} analog.

17. The method of claim 15, wherein said esters of said parent PGF_{2α} analog are selected from the group consisting of methyl esters of said parent PGF_{2α} analog, ethyl esters of said parent PGF_{2α} analog, isopropyl esters of said parent PGF_{2α} analog, glyceryl esters of said parent PGF_{2α} analog, and other similar substituent esters of said parent PGF_{2α} analog.

18. The cosmetic prodrug of claim 11, wherein said FP receptor agonist test compounds in said slowly hydrolyzing form is selected from the group consisting of Bimatoprost in said amide form, Latanoprost in said amide form, Travoprost in said amide form, and mixtures thereof.

19. The cosmetic prodrug of claim 11, wherein said FP receptor agonist test compounds in said rapidly hydrolyzing form is selected from the group consisting of Bimatoprost in said ester form, Latanoprost in said ester form, Travoprost in said ester form, and mixtures thereof.

20. The cosmetic prodrug of claim 18, wherein said FP receptor agonist test compounds in said rapidly hydrolyzing form is selected from the group consisting of Bimatoprost in said ester form, Latanoprost in said ester form, Travoprost in said ester form, and mixtures thereof.
